(19) United States
(12) Patent Application Publication
(10) Pub. No.: US 2020/0165287 A1
(43) Pub. Date: May 28, 2020

(54) SALT OF (SS)-ADENOSYL METHIONINE WITH INOSITOL HEXAPHOSPHATE, AND PROCESS FOR THE PREPARATION THEREOF

(71) Applicant: GNOSIS S.p.A., Milano (IT)

(72) Inventors: Auro Roberto TAGLIANI, Desio (IT); Daniele GREGORI, Antonino (CH); Davide BIANCHI, Desio (IT); Marco BERNA, Desio (IT); Federica COLZANI, Desio (IT)

(73) Assignee: GNOSIS S.p.A., Milano (IT)

(21) Appl. No.: 16/628,624

(22) PCT Filed: Jul. 3, 2018
(86) PCT No.: PCT/EP2018/067882

§ 371 Date: Jan. 3, 2020

(30) Foreign Application Priority Data
Jul. 4, 2017 (IT) 102017000074957

(51) Int. Cl. C07H 19/167; C07H 1/08

(52) U.S. Cl. CPC C07H 19/167; C07H 1/08

(57) ABSTRACT

The invention relates to the salt of (SS)-adenosyl methionine with myo-inositol 1,2,3,4,5,6 hexakisphosphate, and pharmaceutical, nutraceutical or veterinary formulations containing it.

### FIELD OF INVENTION

The invention relates to the salt of (SS)-adenosyl methionine with myo-inositol 1,2,3,4,5,6 hexakisphosphate, and pharmaceutical, nutraceutical or veterinary formulations containing it.

### BACKGROUND OF THE INVENTION

S-Adenosyl-L-methionine (SAMe or ademetionine) is a natural product present in all living organisms, wherein it acts as an important methylating agent in the cell metabolism. In view of its ubiquitous role, a deficiency of this important substance in the human body contributes to the onset of numerous disorders; for example, it is associated with the development of osteoarthritis, cirrhosis of the liver, cystic fibrosis, some depressive states, and aging-associated diseases such as Alzheimer disease and Parkinson disease. Moreover, low levels of SAMe are associated with the development of cardiovascular disorders. SAMe in injectable form is a medicament approved in many European countries, while the oral form is also used as a diet supplement.

SAMe is characterized by strong chemical instability; it breaks down rapidly even at room temperature, mainly giving rise to S-adenosylhomocysteine (SAH), homoserine, methylthioadenosine (MTA), adenine and S-5′-adenosyl-(5′)-3-methylpropylamine (decarboxylated SAMe or deca-SAMe). The product is more stable as the salt of a strong acid; numerous salts of SAMe with strong organic or inorganic acids, including polyacids, are known, such as the salt with polyphosphoric acid described in EP0191133. However, only formulations salified with 1,4-butanedisulphuric acid, sulphuric acid and p-toluenesulphonic acid exist on the market; the form salified with the mixture of sulphuric/p-toluenesulphonic acids is also indicated as SAMe Pates (PTS).

All SAMe salts are more stable when they are very dry; however, they are all hygroscopic and tend to absorb water, which triggers degradation processes, reducing the duration of the active ingredient. Formulations containing SAMe salts must therefore be prepared in premises with controlled humidity, generally with relative humidity levels below 20%.

SAMe also exists in two diastereoisomeric forms: (S)-S-adenosyl-L-methionine (Formula I)

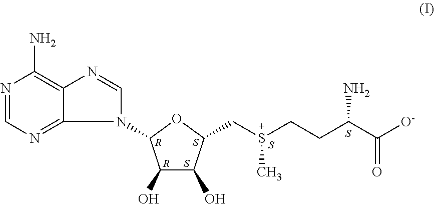

and (R)-S-adenosyl-L-methionine (Formula II)

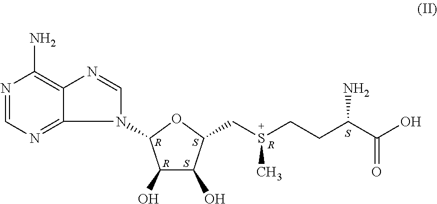

The two isomers can be distinguished by HPLC analysis. The SAME produced by living organisms is biosynthesized using L-methionine as substrate to give a single diastereoisomer, (S)-S-adenosyl-L-methionine, also indicated as (S)-S-adenosylmethionine or (SS)SAMe, which is the pharmacologically useful substance.

The (R) diastereoisomer, also referred to as (R)-S-adenosylmethionine or (SR)SAMe, is a degradation product; (SS)SAMe tends to isomerise until the conditions of equilibrium with the two diastereoisomers in the 1:1 ratio are reached. The (SR) isomer is not only inactive in physiological functions, but also considered potentially harmful (Borchardt and Wu, J. Med. Chem.; 19 (9), 1099, 1976).

SAMe can also be obtained by chemical synthesis, but industrial production usually takes place by fermentation, by means of multistep processes. To obtain the best-quality active ingredient, including the (SS)SAMe content, it is important to control the temperature and pH throughout the manufacturing process, as those are the main factors that influence the isomerization and degradation processes (EP1283845, EP 1071001).

Both isomerization and degradation to other chemical species represent a problem that limits the shelf life of the medicament; isomerization is the most rapid, quickly reducing the dose of (SS)SAMe, while chemical degradation leads to the formation of the above-mentioned compounds, which cause a change of color and the formation of an unpleasant odor, as reported in EP2742943.

Research into new methods of stabilising the active ingredient is still ongoing, confirming that stability of SAMe is still an unsolved problem. For example, U.S. Pat. No. 9,534,010 reports the preparation of the salt with 3-indolylpropionic acid, which is obtained from SAMe Pates or SAMe 1,4-butanedisulphate; however, the percentage of (SS) isomer of the active ingredient obtained is not indicated, and the stability in relation to diastereoisomeric purity is not stated. The analysis method used (UV spectrum in aqueous solution) does not give any indication about the isomerization of the active isomer.

Another method of increasing the stability of SAMe is to use excipients which, when mixed with the SAMe salt, slow its degradation, as reported in EP2742943. Said solution partly eliminates the formation of unpleasant odors, but does not prevent the isomerization of the product and therefore the formation of (SR)SAMe (once again, the published analysis method does not disclose the isomerization of the product). The use of excipients in general obviously leads to formulations with a lower concentration of active ingredient.

Inositol hexaphosphate (IP6) is a natural compound found in the seeds of many plants, especially cereals and legumes, and is often accompanied by the presence of lower homologues (inositol pentaphosphate, inositol tetraphosphate, etc., also indicated as IP5, IP4, etc.) originating from degradation. The mixture of various inositol phosphates is generically indicated as phytic acid, and is available on the market in aqueous solution or as sodium salt; phytic acid is also commercially available in form of its calcium and magnesium mixed salt, called phytin. Phytic acid has chelating properties towards bi- and tri-valent metals, and can be degraded by enzymes called phytases, produced by both plants and microbes, including some normally present in the intestinal flora of humans and other mammals.

A mixed salt or complex of phytic acid comprising SAMe and metal ions (at least one metal), described in EP1896489, contains at least one other cation in addition to SAMe, in particular the ion of an alkali or alkaline-earth metal such as Na, K, Ca, etc. Any compound of inositol having at least one phosphate group, not only the hexaphosphate, is also indicated as phytic acid. In view of the abundant presence of other substances, the active ingredient content does not exceed 25% (SAMe ion). Also in this example, the preparation is effected through an intermediate, another SAMe salt (sulphate, Pates or 1,4-butanedisulphate), so the end product may also contain other cations (sulphates, butanedisulphates or tosylates) deriving from the preparation process, and phosphates deriving from the degradation of inositol hexaphosphate to lower homologues. Once again, the diastereoisomeric purity of the product obtained is not stated, and the stability tests only consider the breakdown of the product, not its isomerization.

A mixture of SAMe Pates with inositol is described in WO2007/080010 as a product for the treatment of depression; in that case, inositol is present at high doses (about 1 g), exceeding those of SAMe (about 100 mg), and can be partly replaced by its prodrug, inositol 1-phosphate. The formulation also requires the presence of magnesium oxide as stabilizer, together with other excipients that considerably dilute the presence of SAMe.

WO 2007/04244 discloses a mixture of SAMe, phytic acid, calcium and magnesium. The disclosed solid compound has no definite formula: possible assumptions for the stoichiometry of the disclosed mixtures correspond to either SAMe₃ Phytate₅ Ca₇ Mg₄ or SAMe₃ Phytate₄ Ca₆ Mg 3 in combination with other SAMe pates salts and impurities. The term “phytic acid” as used in WO 2007/04244 refers to a mixture of inositol-phosphates having different proportions of phosphate groups linked to the sugar moiety. The molar ratio between SAMe and phytic acid is lower than 0.75.

WO 2012035685 discloses a yeast enriched in SAMe and phytic acid. The product is not isolated from the yeast cells. Said document in particular discloses:
a yeast enriched in SAMe, dried and then added with phytic acid, having a SAMe content of about 3%, or
a composition comprising SAMe pates added with phytic acid and other components. The molar ratio between SAMe and phytic acid is about 0.66.

### Definitions

SAMe=S-adenosyl-L-methionine or ademetionine or SAM-e (stereochemistry not specified).
(SS)SAMe=S-adenosyl-L-methionine diastereoisomer (SS), formula I
(SR)SAMe=S-adenosyl-L-methionine diastereoisomer (SR), formula II
Isomeric purity=% ratio between (SS)SAMe and the sum of [(SS)SAMe+(SR)SAMe]
SAMe ion=assay value of a solid or solution expressed as S-adenosyl-L-methionine (stereochemistry not specified) independently of the salification counterion
Inositol hexaphosphate=inositol-hexakisphosphate or myo-inositol (1,2,3,4,5,6)-hexakisphosphate or IP6, formula III.

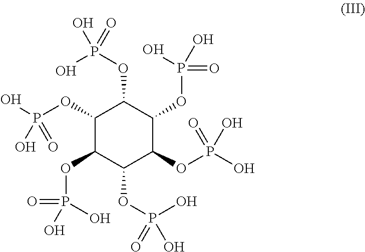 [SMILES: O=P(O)(O)O[C@H]1[C@H](OP(=O)(O)O)[C@@H](OP(=O)(O)O)[C@H](OP(=O)(O)O)[C@@H](OP(=O)(O)O)[C@H]1OP(=O)(O)O]

### DESCRIPTION OF THE INVENTION

It has now been found that the salt of inositol-hexaphosphate (phytic acid) with (SS)-S-adenosyl methionine is particularly stable and well absorbed after oral administration, and characterized by high diastereoisomeric purity. The salt of the invention is surprisingly resistant to degradation by isomerization, thus allowing the formulation of preparations that maintain their biological efficacy over the long term.

Moreover, when administered orally, the enteric absorption of the new salt herein described has proved surprisingly higher compared to other SAMe compounds currently available on the market.

The (SS)SAMe salt of the invention can be obtained directly from yeast, with no need to isolate an intermediate product, leading to economic advantages and to a product of better quality, especially its enantiomeric purity.

The subject of the invention is therefore the (S,S)-S-adenosyl methionine salt with inositol-hexaphosphate wherein the ratio of SAMe to phytic acid is equimolar, i.e. consisting of about 1 mole of inositol hexaphosphate to 1 mole of (SS)SAMe, and typically ranging between 0.75 and 1.0 (mole/mole), preferably from 0.8 to 1.0.

In particular, the diastereoisomeric purity of the salt of the invention is greater than 70% in terms of the pharmacologically active (SS)SAMe enantiomer content, with an (R,S)-S-adenosyl methionine content not exceeding 30%, preferably lower than 15%, and more preferably lower than 5% of the total SAMe.

The SAMe content, expressed as the ion, ranges between 30% and 40%, preferably between 34% and 39%, by weight. The pharmacologically inactive (R,S)-SAMe enantiomer content in the product in question is therefore less than 10%, preferably less than 5%, and even more preferably less than 1% of the total weight.

The salt is devoid or substantially devoid of cations or anions such as Na⁺, Ca⁺⁺, Mg⁺⁺, iron and other alkaline or alkaline earth metals, sulphates, chlorides, p-toluenesulphonates, butanedisulphates, phosphates and other anions of strong organic or inorganic acids, other than inositol phosphates.

“Devoid or substantially devoid” means an ion content of less than 1% by weight, preferably less than 0.1%, and even more preferably less than 0.01% by weight. A pure salt from which other cations and anions are wholly absent, at least at the detectability limits of commonly used analysis techniques, is particularly preferred.

Preferably, the product is almost devoid of inositol pentaphosphate and other lower homologues, with a total quantity of other inositol phosphates (1 to 5 phosphates) of less than 5%, and more preferably less than 1% by weight of the compound.

The invention also relates to the process for the preparation of said (S,S)-S-adenosyl methionine salt directly from the biomass, without isolating other intermediate products. The process uses chromatographic purification techniques and tangential filtration techniques to obtain a purified aqueous solution, from which the product of the invention is isolated by spray-drying, freeze-drying or precipitation with solvents. Moreover, the process is effected under conditions that limit the chemical degradation reactions, in particular the isomerization of (SS)SAMe, so that the quality of the end product is excellent: high overall purity, free from metals and organic and inorganic salts, and high diastereomeric purity.

The process of the invention comprises:
a) production of SAMe from a microbial biomass, preferably a yeast,
b) lysis of said biomass under acidic conditions,
c) separation of the biomass and its fragments from the aqueous solution containing SAMe,
d) purification of the aqueous solution of SAMe by resin chromatography, in one or more steps, using an aqueous solution of inositol hexaphosphate,
e) optionally, decolorizing of the aqueous solution of SAMe with charcoal, diatomaceous earths or other decolorizers,
f) drying of the purified SAMe solution by freeze-drying or spray-drying, or
g) as an alternative to step f), precipitation of the (SS)-S-adenosyl-L-methionine inositol hexaphosphate salt with the use of organic solvents, especially water-miscible organic solvents, preferably methanol, ethanol, isopropanol or acetone.

The starting SAMe can be obtained from a biological source, such as yeast, according to the procedures described in EP1283845. Compliance with the temperature and pH values specified in the various manufacturing steps is crucial to limit the degradation and isomerization of the (SS)SAMe isomer.

The product can be purified by subjecting its aqueous solutions to chromatography processes through resins, using resins based on natural or synthetic polymers, either functionalized (e.g. ion-exchange resins) or non-functionalized (e.g. adsorbent resins). Resins with a polystyrene and polyacrylic matrix, such as those described in the examples below, are particularly preferred for industrial processing.

The product can be isolated by precipitation with solvent, freeze-drying or spray-drying. The latter two technologies are preferred because they do not use organic solvents; however, the quality of the product obtained is excellent in all cases, and does not depend on the technology used. It is important to ensure that the product is dry enough to guarantee good stability; the residual moisture must not exceed 10% of water, preferably less than 5% of residual water, determined by Karl Fischer titration. Residual moisture values even below 1% can be obtained, either directly by the techniques indicated or by secondary drying, for example by placing a solid product in a dryer under vacuum.

When the (SS)-S-adenosyl methionine inositol hexaphosphate of the invention is obtained by spray-drying, it is characterized by a spheroidal particle shape and a particle size of less than 100 microns; by suitably adjusting the operating conditions, a smaller particle size can also be obtained, if necessary under 10 microns. The product presents as a white powder characterized by good flowability, although it is hygroscopic.

The spray-drying process is usually effected in a hot-air flow with an input air temperature ranging from 130 to 170° C., preferably from 140 to 160° C. The output air temperature is regulated in the range between 75 and 110° C., preferably between 85 and 95° C., by suitably varying the operating conditions.

The conditions specified give rise to (SS)SAMe inositol-hexaphosphate in a sufficiently dry solid form, limiting the degradation of the product, including isomerization, to obtain a product of quality equivalent to that obtained by freeze-drying.

The product obtained also combines the best characteristics of spherical shape, particle size and other physical properties, thus providing the product with good flowability and simultaneously a good degree of packing. This allows its use in tablet presses and filling machines for vials, sachets, capsules and other dosage forms.

The flowability and particle-size characteristics of the powder are of crucial importance to guarantee an accurate dose, as a powder with poor flowability and/or an irregular shape may not completely fill the chamber, thus making the dose imprecise.

In the specific case of SAMe salts, there is the additional problem of hygroscopicity, leading to downtime for cleaning the formulation machines, and therefore reduced productivity of the plant. This problem affects all the SAMe salts known to date, which require dehumidified premises with relative humidity (RH) of less than 20% for the fractionation and batching operations; such levels of dryness are only obtainable with specific technologies, such as Munters dryers, which involve additional costs. In the case of the salt with inositol hexaphosphate, the problem is less severe, because the product, though hygroscopic, does not become sticky, and remains flowable. It can therefore be manufactured in premises which are not particularly dry, with relative humidity values reachable by a normal air-conditioning installation (e.g. RH>40%), with a reduction in manufacturing costs due to the higher productivity of the machinery and lower plant costs.

Another aspect of the invention also relates to a mixture of a pharmacologically acceptable SAMe salt rich in (SS) diastereoisomer and inositol hexaphosphate or pharmacologically acceptable salts thereof for use in pharmaceutical, nutraceutical or veterinary formulations wherein the molar ratio of SAMe to inositol hexaphosphate is less than 1.

The following examples illustrate the invention in greater detail.

### Example 1

A solution of pure phytic acid (IP6) is prepared from commercial inositol hexaphosphate sodium salt (pure Nal2 inositol hexaphosphate) which is dissolved in water; the solution is then loaded into an Amberlite IRA1200H ion-exchange resin column pre-activated in acid form, and the resin is washed with demineralized water. All the outgoing fractions from the column which have a pH of less than 2 are collected, to obtain a solution consisting of over 90% inositol hexaphosphate and minimal percentages of inositol pentaphosphate; inositol tetraphosphate and other lower homologues are almost absent. The solution is stored cold in plastic containers.

1000 kg of Saccharomyces carlsbergensis yeast biomass is enriched in SAMe content by adding 100 kg of yeast cream, 100 liters of water, 2 kg of D,L-methionine, 12 kg of glucose monohydrate and 1.5 kg of citric acid, which are then maintained in fermentation for 2 hours at +27° C., blowing in sterile air. At the end of fermentation the biomass is cooled to +12° C., and sulphuric acid is added until pH 1.2 is reached. The biomass is then subjected to mechanical cell disruption with the Constant Cell Disruption System (Constant System Ltd.), keeping the temperature controlled between +2° C. and +12° C. The cell lysate is subjected to microfiltration to separate the particulate matter, while the supernatant is loaded into an IRC86 resin column (Rhom&Haas), maintaining the controlled temperature. The resin is washed with demineralized water and acetic acid, and the product is then eluted from the resin using the inositol hexaphosphate solution prepared as described above.

The aqueous acid solution of SAMe-inositol hexaphosphate is then loaded onto a Resindion 825L resin, and a clear solution of (SS)SAMe inositol hexaphosphate with purity exceeding 96% is obtained.

The aqueous solution is then concentrated by ultrafiltration, and subsequently by distillation under vacuum, to reach about 100 g/l; the exact concentration of SAMe (ion) and inositol hexaphosphate is determined, and adjusted to an equimolar ratio. The concentrate is then dried in a spray-drying unit by atomization with hot air at +160° C., and a product with a humidity level lower than 4% (determined by Karl-Fisher titration) is collected.

A white powder with a SAMe ion content of 39% is obtained, 95% of which consists of the (SS) isomer and 5% of the (SR) isomer. The total impurities present are under 5% (HPLC area).

### Example 2

(SS)S-Adenosyl methionine is produced by biotransformation with yeast as described in Example 1. The (SS)SAMe-enriched yeast is subjected to thermoacid lysis by adding inositol hexaphosphate to pH 2.0 and heating at +80° C. for a few seconds, followed by rapid cooling to temperatures lower than +12° C. The suspension thus obtained is subjected to microfiltration, and the resulting permeate then undergoes chromatographic purification at controlled temperature as described in Example 1, until a concentrated solution of (SS)SAMe inositol hexaphosphate at a concentration of about 100 g/l, with an approximately equimolar ratio between IP6 and SAMe, is obtained.

The solution is then spray-dried by atomization in a chamber heated to +140° C., under flow conditions such that the temperature of the outgoing product is about +90° C.

A white powder with a 34% SAMe ion content, 4% residual moisture and 85% enantiomeric purity as (SS)SAMe is obtained.

The Na, Fe, Ca and Mg ion content of the product is less than 0.1% for each element, while the total heavy metals (titrated as specified in Ph. Eur. 2.4.8, method A) are below 10 ppm. The sulphate, chloride and phosphate content is less than 0.1% per anion. Over 90% of the phytic acid present in the product consists of inositol hexaphosphate.

### Example 3

The process is effected as described in Example 2, but using sulphuric acid for the thermoacid lysis of the yeast. The purification of the (SS)SAMe solution proceeds as described in Example 1, but using the aqueous solution of inositol hexaphosphate for the elution of the product from IRC86 resin and for all subsequent manufacturing steps, which are effected cold.

The resulting solution is tested for SAMe ion content, and the phytic acid solution is adjusted to a molar ratio of 1.02 (moles of SAMe ion/moles of inositol hexaphosphate).

The product is isolated by spray-drying, and a white powder with a 39.5% SAMe ion content, 3.3% residual moisture and 78.4% enantiomeric purity is obtained.

The inorganic anion and cation content is very similar to that described in Example 2, including sulphates; over 90% of the SAMe counterion consists of inositol hexaphosphate.

### Example 4

The process is effected as described in Example 2, but using hydrochloric acid for the thermoacid lysis of the yeast. The purification of the (SS)SAMe solution proceeds as described in Example 3, using phytic acid for all subsequent manufacturing steps, which are effected cold.

The SAMe content is determined by HPLC analysis, and IP6 is added until a molar ratio of SAMe ion to inositol-6-phosphate amounting to 0.9 moles/moles is reached.

The product is isolated by spray-drying, and a white powder with a 34% SAMe ion content, 2.5% residual moisture and 90% enantiomeric purity is obtained.

The inorganic anion and cation content is very similar to that described in Example 2, including chlorides; over 90% of the SAMe counterion consists of inositol hexaphosphate.

### Example 5

A 7% solution of Na₁₂ inositol hexaphosphate in water is prepared, and subjected to electrodialysis treatment to obtain the corresponding solution of inositol hexaphosphate acid (IP6).

The process is effected as described in Example 4, but using sulphuric acid for the thermoacid lysis of the yeast. The purification of the (SS)SAMe solution proceeds as described in Example 2, using the IP6 acid solution for all subsequent manufacturing steps, which are effected cold.

The solution is decolorized with activated carbon, after which the SAMe ion content is determined by HPLC analysis and IP6 is added to obtain a molar ratio of 0.76 (SAME ion/inositol hexaphosphate).

The product is isolated by spray-drying, and a white powder with a 31.2% SAMe ion content, 2.5% residual moisture and 98% enantiomeric purity is obtained.

### Example 6

Comparative Example

The process is effected as described in EP1896489 to prepare a sample of SAMe phytate. Powdered commercial SAMe 1,4-butanedisulphate is used, and processed as described in Example 3 of EP1896489: dissolution in water, addition of phytic acid, incubation in ice, addition of ethanol, and filtration under vacuum.

The white precipitate obtained is filtered and dried under vacuum. The resulting product has 1.45% residual moisture, a 25.6% SAMe ion content and 98.45% purity. Its enantiomeric purity is 76.96% as (SS)SAMe isomer. The product is called 04B17DS.

### Example 7

Comparative Example

The product obtained as described in Example 6 (comprising SAMe and phytic acid) and a sample of SAMe inositol hexaphosphate obtained as described in Example 5 are subjected to an accelerated stability test, incubated in a thermostatic stove at +55° C., and the analyses are repeated after 5 days.

The samples are analysed for the following values:
SAMe ion content (total of all enantiomers) determined by HPLC and expressed as % by weight
enantiomeric purity, determined by HPLC and expressed as % of (SS) isomer
active ingredient content, calculated by multiplying the previous two values and expressed as % by weight of (SS)SAMe
overall purity, determined by HPLC as SAMe area/total area of chromatogram, expressed as area %. The figure comprises the values of all known degradation impurities.

The results shown in the Table are obtained.

| | |
|---|---|
| Starting value | Final value |

04B17DS

| | | |
|---|---|---|
| SAMe content | 25.6% w/w | 17.7% w/w |
| (SS) enantiomer | 76.96% | 50.66% |
| (SS)SAMe content | 19.70% | 8.95% |
| Purity (% area) | 98.45% | 90.15% |

AT1003

| | | |
|---|---|---|
| SAMe content | 34.45% w/w | 30.6% w/w |
| (SS) enantiomer | 77.2% | 69.56% |
| (SS)SAMe content | 26.59% | 21.28% |
| Purity (% area) | 95.98% | 90.0% |

The data in the Table clearly show that, conditions being equal, sample AT1003 maintains a higher content of the active ingredient (SS)SAMe after the stress test. The degradation reactions to give MTA, SAH and other known impurities, globally expressed by the purity data in area %, are comparable for the two samples. However, the reaction of degradation by isomerization is surprisingly lower for sample AT1003, which maintains an excess of the (SS)SAMe form, whereas sample 04B17DS is completely isomerized.

The greater stability of the active ingredient in AT1003 is therefore mainly due to greater resistance to isomerization, rather than to other degradation reactions.

### Example 8

Pharmacokinetic Profile in the Rat

70 Sprague Dawley rats (35 male and 35 female), aged 7-9 weeks and weighing 176-200 g, were acclimatized in a cage for two weeks before the start of the treatment, maintained at +22° C. and 55% relative humidity. The rats were fed on 4RF 21 (Mucedola) feed, and weighed before the beginning and at the end of the test. At the time of treatment, 32 animals per group (16 males and 16 females) were selected from those with the most similar weights, and assigned to the two treatments.

The SAMe inositol hexaphosphate (batch 1500282: SAMe ion content 37.48%, isomeric purity as (SS)SAMe: 77.2%) was compared with a commercial sample of SAMe Pates (mixed sulphate/p-toluenesulphate salt), at the same doses (expressed as SAMe ion).

The product was administered by cannula, dissolved in water, at two dose levels, namely 134 mg/kg and 95 mg/kg. The rats were fasted overnight before administration and for a further two hours after administration. The two equimolar doses guaranteed an equal intake of SAMe ion.

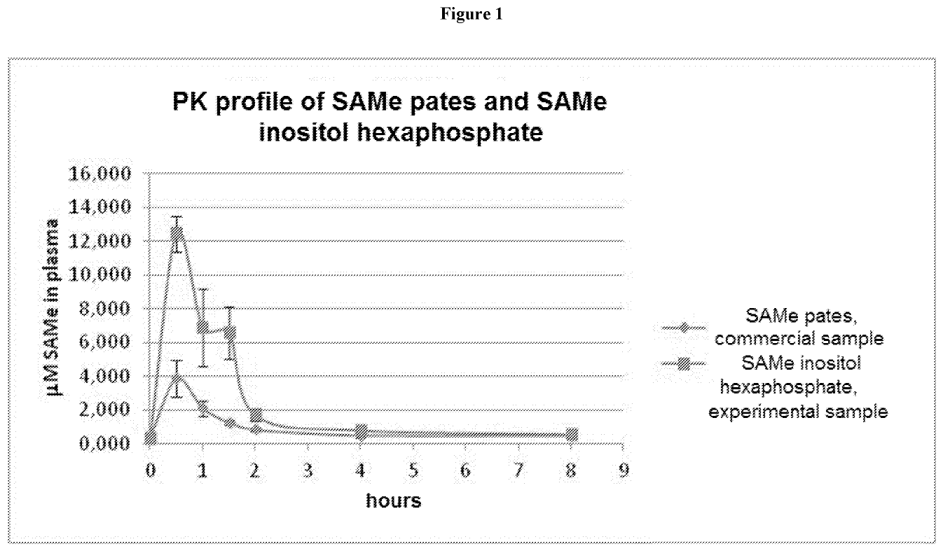

The blood levels of SAMe were assayed by taking a sample from the tail vein at fixed intervals, at times 0 (pre-administration), 0.5, 1, 1.5, 2, 4, 8 and 24 hours after administration. The blood samples were centrifuged after addition of EDTA to separate the plasma from the cell fraction. The plasma was then analysed by HPLC according to the method reported in Wise, Fullerton J. Liq. Chromatogr. 18 (1995) 2005-17, and the data shown in the graph in FIG. 1 (mean of all samples) were obtained.

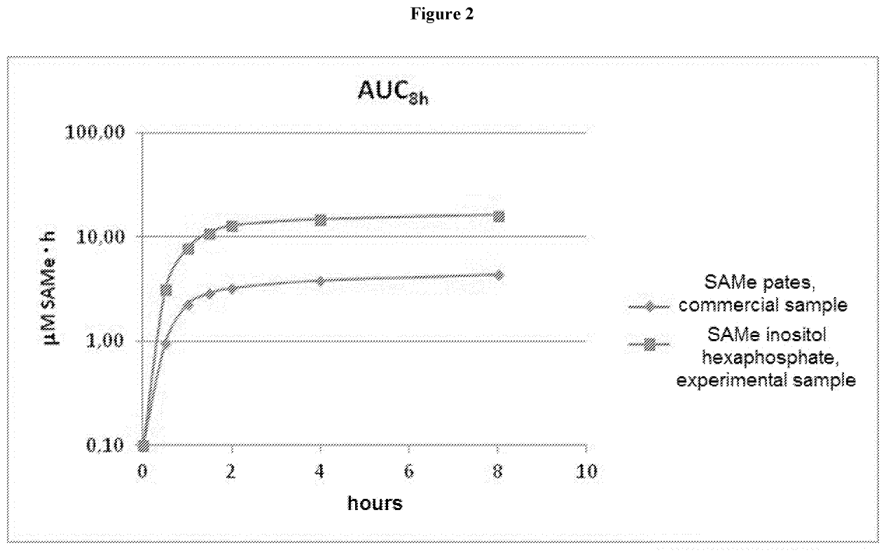

The SAMe plasma concentration values are also used to determine, according to the Area Under Curve (AUC) calculation, the total quantity of active ingredient absorbed, obtaining the values set out in FIG. 2.

As the figures clearly show, salification with inositol hexaphosphate (SAMe IP6) leads to better absorption and a higher plasma concentration of active ingredient than the product currently on the market (SAMe Pates).

### Example 9

Comparative Example

Some samples of the products obtained as described in the previous examples are subjected to stability tests, storing them at +25° C., in a thermostatic stove.

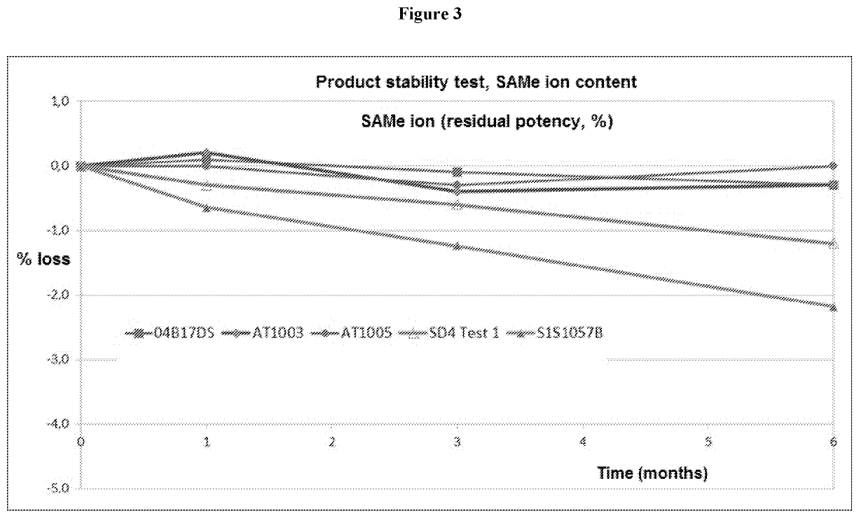

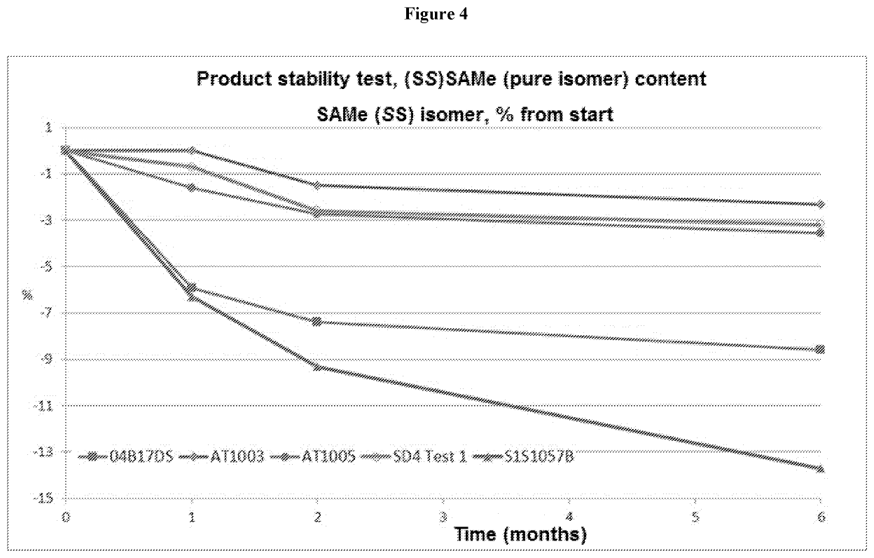

The SAMe ion content analysis (FIG. 3) and the diastereomeric purity relative to the (SS) isomer (FIG. 4) are periodically repeated, and the result is compared with the starting value for the same sample, obtaining the results reported.

The data obtained with the product obtained according to EP 1896489 and two commercial samples of SAMe, 1,4-butanedisulphate salt (SD4 Test 1) and Pates salt (batch S1S1057B), are compared.

If only the SAMe ion content is observed, all samples are stable (FIG. 3).

As clearly shown in FIG. 4, the content of the active ingredient, (SS)SAMe isomer, is stable for longer in the case of the inositol hexaphosphate salt (samples AT1003 and AT1005) and the commercial 1,4-butanedisulphonate salt (sample SD4 Test 1). The commercial Pates salt (S1S1057B) and the SAMe-metal-phytate complex obtained according to EP1896489 (sample 04B17DS) lose the (SS)SAMe diastereoisomer more quickly, thus reducing the quantity of active ingredient.

1. (SS)-S-Adenosyl-L-methionine inositol hexaphosphate salt wherein the molar ratio of SAMe to inositol-hexaphosphate ranges from 0.8 to 1.0.

2. (SS)-S-Adenosyl-L-methionine inositol-hexaphosphate salt according to claim 1 wherein the ratio of SAMe to inositol hexaphosphate is equimolar.

3. (SS)-S-Adenosyl-L-methionine inositol hexaphosphate salt according to claim 1 wherein the salt has a (SS)-S-Adenosyl-L-methionine ion content exceeding 30% by weight.

4. (SS)-S-Adenosyl-L-methionine inositol hexaphosphate salt according to claim 3 wherein the salt has a (SS)-S-Adenosyl-L-methionine ion content ranging from 30% to 40% by weight.

5. (SS)-S-Adenosyl-L-methionine inositol hexaphosphate salt according to claim 4 wherein the salt has a (SS)-S-Adenosyl-L-methionine ion content ranging from 34% to 39% by weight.

6. (SS)-S-Adenosyl-L-methionine inositol hexaphosphate salt according to claim 1, wherein the salt has isomeric purity exceeding 70%.

7. (SS)-S-Adenosyl-L-methionine inositol hexaphosphate salt according to claim 1, wherein the salt has isomeric purity exceeding 95%.

8. (SS)-S-Adenosyl-L-methionine inositol hexaphosphate salt according to claim 1 wherein the salt is devoid or substantially devoid of inorganic cations and inorganic or organic anions.

9. (SS)-S-Adenosyl-L-methionine inositol hexaphosphate salt according to claim 8, wherein the salt is devoid or substantially devoid of alkali and alkaline-earth metals, iron, sulphates, p-toluenesulphonates, phosphates, chlorides and 1,4-butanedisulphates.

10. (SS)-S-Adenosyl-L-methionine inositol hexaphosphate salt according to claim 1 wherein the content of inositol pentaphosphate or other inositol-phosphates (1 to 4 phosphates) is globally lower than 5% by weight of the salt.

11. A process for the preparation of (SS)-S-adenosyl-L-methionine inositol-hexaphosphate salt of claim 1, comprising:
a) production of SAMe from a microbial biomass, preferably a yeast,
b) lysis of said biomass under acidic conditions,
c) separation of the biomass and fragments thereof from the aqueous solution containing SAMe,
d) purification of the SAMe aqueous solution by resin chromatography, in one or more steps, using an inositol hexaphosphate aqueous solution,
e) optionally, decolorization of the SAMe aqueous solution with charcoal, diatomite or other decolorizing agents,
f) drying the purified SAMe solution by freeze-drying or spray-drying, or
g) alternatively to step f), precipitation of (SS)-S-adenosyl-L-methionine inositol hexaphosphate salt using water-miscible organic solvents, selected from the group consisting of methanol, ethanol, isopropanol and acetone.

12. A pharmaceutical, nutraceutical or veterinary formulation comprising (SS)-S-adenosyl-L-methionine inositol-hexaphosphate salt of any one of claims 1-10 and a pharmaceutically, nutraceutically, or veterinary acceptable excipient.

13. A pharmaceutical, nutraceutical or veterinary formulation comprising a mixture of a pharmacologically acceptable SAMe salt enriched in (SS) diastereomer and inositol hexaphosphate or pharmacologically acceptable salts thereof, wherein the molar ratio of SAMe to inositol hexaphosphate ranges from 0.8 to 1.0.
